(19) United States
(12) Patent Application Publication
(10) Pub. No.: US 2026/0355730 A1
(43) Pub. Date: Nov. 12, 2026

(54) PROCESS FOR MAKING CATALYST FOR THE PRODUCTION OF METHYL METHACRYLATE BY OXIDATION ESTERIFICATION

(71) Applicants: Dow Global Technologies LLC, Midland, MI (US); Rohm and Haas Company, Collegeville, PA (US)

(72) Inventors: Wen-Sheng Lee, Midland, MI (US); Kirk W. Limbach, Dresher, PA (US); Victor J. Sussman, Midland, MI (US); Alexey Kirilin, Terneuzen (NL)

(21) Appl. No.: 19/125,802

(22) PCT Filed: Nov. 20, 2023
(86) PCT No.: PCT/US2023/080435

§ 371 Date: Apr. 30, 2025

Related U.S. Application Data
(60) Provisional application No. 63/431,037, filed on Dec. 8, 2022.

(51) Int. Cl. B01J 37/02; B01J 21/08; B01J 21/14; B01J 23/02; B01J 23/52; B01J 23/66; B01J 23/68; B01J 23/89; B01J 27/224; B01J 35/45; B01J 37/00; B01J 37/04; B01J 37/06; B01J 37/08; B01J 37/16; C07C 67/40; C07C 69/54

(52) U.S. Cl. CPC B01J 37/0203; B01J 21/08; B01J 21/14; B01J 23/02; B01J 23/52; B01J 23/66; B01J 23/687; B01J 23/688; B01J 23/8946; B01J 27/224; B01J 35/45; B01J 37/009; B01J 37/0236; B01J 37/04; B01J 37/06; B01J 37/088; B01J 37/16; C07C 67/40; C07C 69/54

(57) ABSTRACT

A process for making a catalyst for oxidative esterification of methacrolein to methyl methacrylate comprises providing a support selected from an oxide of silicon, a carbide of silicon, a metal oxide, and a metal carbide. Particles of at least one oxide of a metal are provided on the support. The support is contacted with a gold salt and heated at a temperature ranging from 50 °C to 600 °C for a time ranging from at least 0.1 h to 48 h to convert the gold salt to gold nanoparticles having an average diameter of less than 12 nm and a standard deviation of +/−4 nm, where at least 75% by number of the gold nanoparticles are positioned within 20 nm of a particle of the oxide of the metal.

### BACKGROUND OF THE INVENTION

The invention relates to a process for making a catalyst for preparing methyl methacrylate from the oxidative esterification of methacrolein.

Heterogeneous catalysts for use in producing carboxylic esters, including methyl methacrylate, from aldehydes are known.

U.S. Pat. No. 8,461,373 discloses a catalyst comprising oxidized nickel and at least one element selected from nickel, palladium, platinum, ruthenium, gold, silver, and copper. The diameter of the catalyst ranges from 10 to 200 μm.

WO 2016/113106 discloses a catalyst comprising gold, silicon oxide, aluminum oxide, and an oxide of at least one element selected from alkali metals, alkaline earth metals, lanthanides having atomic numbers from 57 to 71, Y, Sc, Ti, Zr, Cu, Mn, Pb and Bi. The mean diameter of the catalyst ranges from 10 to 200 μm.

However, there is a need for an improved catalyst and process for production of catalyst that may improve selectivity and/or reduce the formation of byproducts.

### SUMMARY OF THE INVENTION

One aspect of the present invention relates to a process for making a catalyst for oxidative esterification of methacrolein to methyl methacrylate. The process comprises providing a support selected from an oxide of silicon, a carbide of silicon, a metal oxide, and a metal carbide. Particles of at least one oxide of a metal are provided on the support. The metal of the at least one oxide of a metal is selected from the group consisting of aluminum, titanium, lanthanides, zirconium, nickel, cobalt, zinc, tellurium, antimony, bismuth, alkali metals, and alkaline earth metals. The support is contacted with a gold salt and then heated at a temperature ranging from 50 °C to 600 °C for a time ranging from at least 0.1 h to 48 h to convert the gold salt to gold nanoparticles having an average diameter of less than 12 nm and a standard deviation of +/−4 nm. At least 75% by number of the gold nanoparticles are positioned within 20 nm of a particle of the oxide of the metal.

### DETAILED DESCRIPTION OF THE INVENTION

All percentage compositions are weight percentages (wt %), and all temperatures are in °C, unless otherwise indicated. Averages are arithmetic averages unless otherwise indicated. The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters. Unless otherwise stated, the average diameter of a particle refers to the average diameter of the particle after the catalyst has been prepared and before the catalyst has been used. An aged catalyst is a catalyst that has been used.

The catalyst of the present invention comprises a support with nickel oxide and gold particles disposed on the support.

The present invention relates to a process for making a catalyst for oxidative esterification of methacrolein to methyl methacrylate.

The process comprises providing a support and providing particles of at least one oxide of a metal on a surface of the support, contacting the support with a gold salt, and heating the support at a temperature ranging from 50 °C to 600 °C for a time ranging from at least 0.1 h to 48 h to convert the gold salt to gold nanoparticles having an average diameter less than 12 nm and a standard deviation of +/−4 nm, wherein at least 75% by number of the gold nanoparticles are positioned within 20 nm of a particle of the oxide of the metal

The support comprises a material selected from an oxide of silicon, a carbide of silicon, a metal oxide, and a metal carbide. The metal of the metal oxide and the metal carbide may be selected from, for example, aluminum, titanium, zirconium, zinc, and magnesium. Preferably, the support is capable of withstanding long-term use in an oxidative esterification reactor. Materials that are capable of withstanding prolonged use are able to avoid being crushed or pulverized during use.

In one embodiment, the support may have an average diameter ranging from 50 nm to 500 μm, preferably from 100 nm to 400 μm, and more preferably from 150 nm to 300 μm. In this embodiment, the catalyst may be suitable for a reactor in which the catalyst is maintained in suspension, such as in a slurry reactor or continuously stirred tank reactor. When the support is less than 500 μm, the support may be directly formed as a reaction product followed by drying, by spray drying the support, by precipitation followed by filtration and/or centrifugation, or by crushing a larger material to form the desired size. Alternatively, the support may be prepared by classifying particles to sort out the desired average particle diameter.

In another embodiment, the support may have an average diameter greater than 500 μm, such as from 500 μm to 10 mm. In embodiments where the average diameter of the support is greater than 500 μm, the catalyst may be used in a reactor comprising a fixed bed, such as, for example, a fixed bed reactor, a trickle bed reactor, or a packed bubble column reactor. For supports having an average diameter greater than 500 μm, the support may be produced by extrusion or pelletization, or any other known method. When forming the support by extrusion or pelletization, the support may be formed with our without a binder.

Particles of at least one metal oxide are provided on the support, where the metal is selected from aluminum, titanium, lanthanides, zirconium, alkali metals, alkaline earth metals, nickel, cobalt, zinc, magnesium, tellurium, antimony, rhenium, tungsten, and bismuth. The particles of at least one metal oxide may be formed on the support by first contacting the support with a salt of the metal and then oxidizing the metal in an environment comprising an oxygen-containing gas, e.g., air or oxygen.

Preferably, the support comprises, consists of, or consists essentially of an oxide of silicon. More preferably, the support comprises, consists of, or consists essentially of an oxide of silicon modified with particles of titanium oxide or nickel oxide. As used herein with respect to the support, the phrase “consists essentially of” excludes the presence of materials that would degrade the mechanical strength of the support. Alternatively, “consists essentially of” means that the support comprises at least 95 wt % of the stated material with respect to the total weight of the support.

Preferably, the support has a surface area greater than 10 m²/g, preferably greater than 30 m²/g, preferably greater than 50 m²/g, preferably greater than 100 m²/g, preferably greater than 120 m²/g.

Preferably, the aspect ratio of the catalyst particle is no more than 10:1, preferably no more than 5:1, and preferably no more than 3:1. Although the shape is not limited, preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

To form gold nanoparticles on the support, the support is contacted with a gold salt, preferably in the form of an aqueous solution comprising the gold salt. The gold salt is then converted to particles of metallic gold by heating the support at a temperature ranging from 50 °C to 600 °C for a time ranging from 0.1 h to 48 h.

In one embodiment, the step of heating the support to convert the gold salt to metallic gold may be conducted in the presence of an oxygen-containing gas, such as air or oxygen, to calcine the catalyst. Calcination may be conducted at a temperature ranging from 150 °C to 500 °C, preferably from 200 °C to 450 °C. Calcination is a preferred process for converting the gold salt to metallic gold because it can simultaneously form the particles of a metal oxide from metal salts.

In an alternative embodiment, the step of heating the support to convert the gold salt to metallic gold may be conducted in the presence of a reducing gas comprising at least 0.1% by volume of a reducing agent.

In another alternative embodiment, the step of heating the support to convert the gold salt to metallic gold may be conducted in the presence of an inert atmosphere, in which case the inert gas allows the gold salt to self-reduce to metallic gold.

In yet another alternative embodiment, the step of heating the support to convert the gold salt to metallic gold may be conducted in the liquid phase in the presence of a solvent and a reductant, wherein the ratio of reductant to solvent is at least 0.01. In this embodiment, the support is heated at a temperature ranging from 50 °C to less than the boiling point of the solvent.

Contacting support with a gold salt may be performed by several different methods. For example, contacting the support with a gold salt may be performed by impregnating the support with an aqueous solution comprising the gold salt, dip coating the support with a solution comprising the gold salt, spray coating the support with a solution comprising the gold salt, or sequentially impregnating the support by impregnating the support with a first solution, either aqueous or organic, to fill at least 80% by volume of any pores that may be present in the support, followed by impregnating the support with a second solution comprising the gold salt. When contacting the support with a solution or aqueous solution of a gold salt, the support may first be dried before the support is heated to convert the gold salt to metallic gold particles.

Preferably, the catalyst is produced by precipitating the gold and metal (i.e., the metal of the metal oxide) from an aqueous solution of metal salts in the presence of the support. In one preferred embodiment, the catalyst is produced by contacting an aqueous solution of a suitable gold precursor salt and nickel salt with a porous inorganic oxide such that the pores are filled with the solution and the water is then removed by drying. The resulting material is then converted into a finished catalyst by calcination or reduction, to decompose the gold salts and metal salts into gold and metal oxides. Preferably, a C₂-C₁₈ thiol comprising at least one hydroxyl or carboxylic acid substituent is present in the solution. Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. Especially preferred thiol compounds include thiomalic acid, 3-mercaptopropionic acid, thioglycolic acid, 2-mercaptoethanol and 1-thioglycerol, including their conjugate bases.

The metal oxide and gold particles are preferably disposed on an outer surface of a support material. Preferably, at least 75% by weight of the gold particles are within an outer 25% of the volume of the catalyst. More preferably, at least 80%, still more preferably at least 85%, by weight of the gold particles are within an outer 25% of the volume of the catalyst.

The metal oxide disposed on the support is preferably in the form of nanoparticles.

Preferably, at least 75% of the gold particles by number of gold particles are within at least 20 nm of a metal oxide nanoparticle. As used herein, the phrase “within at least X nm” means that an edge of a gold particle is within X nm of an edge of the metal oxide nanoparticle nearest the gold particle. Preferably, at least 75% of the gold particles are within at least 15 nm of a metal oxide nanoparticle, more preferably within at least 12 nm of a metal oxide nanoparticle, and even more preferably within at least 10 nm of a metal oxide nanoparticle.

More preferably, at least 75% of the gold particles by number of the gold particles are within at least 20 nm of two metal oxide nanoparticles, i.e., an edge of the gold particle is within at least 20 nm of an edge of the two metal oxide nanoparticles nearest the gold particle. Preferably, at least 75% of the gold particles are within at least 15 nm of two metal oxide nanoparticles, more preferably within at least 12 nm of two metal oxide nanoparticles, and even more preferably within at least 10 nm of two metal oxide nanoparticles.

Even more preferably, at least 75% of the gold particles by number of the gold particles are within at least 20 nm of at least three metal oxide nanoparticles, i.e., an edge of the gold particle is within at least 20 nm of an edge of at least the three metal oxide nanoparticles nearest the gold particle. Preferably, at least 75% of the gold particles are within at least 15 nm of at least three metal oxide nanoparticles, more preferably within at least 12 nm of at least three metal oxide nanoparticles, and even more preferably within at least 10 nm of at least three metal oxide nanoparticles.

The gold particles have an average diameter of less than 12 nm, preferably less than 10 nm, and more preferably less than 8 nm. The standard deviation of the average diameter of the gold particles is +/−4 nm, preferably +/−2.5 nm. As used herein, the standard deviation is calculated by the following equation:

$$standard deviation = \sqrt{\frac{{\sum }_{i = 1}^{n} \vert x - \overline{x} \vert}{n}}$$

where x is the size of each particle, x̅ is the mean of the n number of particles, and n is at least 500.

The metal oxide nanoparticles preferably have an average diameter of less than 5 times the average diameter of the gold particles, more preferably an average diameter of less than 4 times the average diameter of the gold particles, even more preferably an average particle diameter of less than 3 times the average diameter of the gold particles, still more preferably an average particle diameter of less than 2 times the average diameter of the gold particles, and yet more preferably an average particle diameter of less than 1.5 times the average diameter of the gold particles. Preferably, the metal oxide nanoparticles have an average diameter at least the half the average diameter of the gold particles, and more preferably at least the same as the average diameter of the gold particles.

The amount by weight of the gold particles with respect to the amount of the metal oxide nanoparticles may range from 1:1 to 1:20. Preferably, the weight ratio of gold particles to metal oxide nanoparticles ranges from 1:2 to 1:15, more preferably, from 1:3 to 1:10, and even more preferably, from 1:3 to 1:6.

The amount by weight of the metal oxide with respect to the amount by weight of the gold particles may range from 0.1:1 to 10:1, preferably from 0.2:1 to 5:1, more preferably from 0.33:1 to 3:1, and still more preferably from 0.5:1 to 2:1.

Preferably, the gold particles are evenly distributed among the metal oxide nanoparticles. As used herein, the term “evenly distributed” means the gold particles are randomly dispersed among the metal oxide nanoparticles with substantially no agglomeration of the gold particles, e.g., less than 10% by weight of the gold particles based on the total weight of the gold particles are in physical contact with another gold particle. Preferably, less than 7.5% by weight of the gold particles based on the total weight of the gold particles are in physical contact with another gold particle, and more preferably, less than 5% by weight of the gold particles based on the total weight of the gold particles are in physical contact with another gold particle.

Preferably, at least 75 wt % of the gold particles are in the outer 50% of the catalyst volume (i.e., the volume of an average catalyst particle), more preferably the outer 40% of catalyst volume, even more preferably the outer 30%, and still more preferably the outer 25. Preferably, the outer volume of any particle shape is calculated for a volume having a constant distance from its inner surface to its outer surface (the surface of the catalyst particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the gold particles are in the outer volume of the catalyst, preferably at least 97 wt %, preferably at least 99 wt %. Preferably, at least 90 wt % (preferably at least 95 wt %, preferably at least 97 wt %, preferably at least 99 wt %) of the gold particles are within a distance from the surface that is no more than 30% of the catalyst diameter, preferably no more than 25%, preferably no more than 20%, preferably no more than 15%, preferably no more than 10%, preferably no more than 8%. Distance from the surface is measured along a line which is perpendicular to the surface. Preferably, the gold particles form an eggshell structure on the support particles. The eggshell layer may have a thickness of 500 microns or less, preferably 250 microns or less, and more preferably 100 microns or less.

Preferably, at least 0.1% by weight of the total weight of the gold particles are exposed on a surface of the catalyst. As used herein, the term “exposed” means that at least a portion of the gold particle is not covered by another gold particle or metal oxide nanoparticle, i.e., the reactants can directly contact the gold particle. The gold particles may therefore be disposed within a pore of the support material and still be exposed by virtue of the reactant being able to directly contact the gold particle within the pore. More preferably, at least 0.25% by weight of the total weight of the gold particles are exposed on the surface of the catalyst, even more preferably, at least 0.5% by weight of the total weight of the gold particles are exposed on the surface of the catalyst, and still more preferably, at least 1% by weight of the total weight of the gold particles are exposed on the surface of the catalyst.

Preferably, the amount of gold as a percentage of the gold and the support is from 0.2 to 5 wt %, preferably at least 0.5 wt %, preferably at least 0.8 wt %, preferably at least 1 wt %, preferably at least 1.2 wt %; preferably no more than 4 wt %, preferably no more than 3 wt %, preferably no more than 2.5 wt %.

Preferably, the process for producing methyl methacrylate (MMA) is performed in an oxidative esterification reactor (OER). The catalyst particles may be present in a slurry or in a catalyst bed, preferably a catalyst bed. The catalyst particles in the catalyst bed typically are held in place by solid walls and by screens or catalyst support grids. In some configurations, the screens or grids are on opposite ends of the catalyst bed and the solid walls are on the side(s), although in some configurations the catalyst bed may be enclosed entirely by screens. Preferred shapes for the catalyst bed include a cylinder, a rectangular solid and a cylindrical shell; preferably a cylinder. The OER further comprises a liquid phase comprising methacrolein, methanol and MMA and a gaseous phase comprising oxygen. The liquid phase may further comprise byproducts, e.g., methacrolein dimethyl acetal (MDA) and methyl isobutyrate (MIB). Preferably, the liquid phase is at a temperature from 40 to 120 °C; preferably at least 50 °C, preferably at least 60 °C; preferably no more than 110 °C, preferably no more than 100 °C. Preferably, the catalyst bed is at a pressure from 0 to 2000 psig (101 kPa to 14 MPa); preferably no more than 2000 kPa, preferably no more than 1500 kPa.

The OER typically produces MMA, along with methacrylic acid and unreacted methanol. Preferably, methanol and methacrolein are fed to the reactor in a methanol:methacrolein molar ratio from 1:10 to 100:1, preferably from 1:2 to 20:1, preferably from 1:1 to 10:1. Preferably, a catalyst bed further comprises inert or acidic materials above and/or below the catalyst. Preferred inert or acidic materials include, e.g., alumina, clay, glass, silica carbide and quartz. Preferably, the inert or acidic material has an average diameter equal to or greater than that of the catalyst, preferably no greater than 20 mm. Preferably, the reaction products are fed to a methanol recovery distillation column which provides an overhead stream rich in methanol and methacrolein; preferably this stream is recycled back to the OER. The bottoms stream from the methanol recovery distillation column comprises MMA, MDA, methacrylic acid, salts and water. In one embodiment of the invention, MDA is hydrolyzed in a medium comprising MMA, MDA, methacrylic acid, salts and water. MDA may be hydrolyzed in the bottoms stream from a methanol recovery distillation column; said stream comprising MMA, MDA, methacrylic acid, salts and water. In another embodiment, MDA is hydrolyzed in an organic phase separated from the methanol recovery bottoms stream. It may be necessary to add water to the organic phase to ensure that there is sufficient water for the MDA hydrolysis; these amounts may be determined easily from the composition of the organic phase. The product of the MDA hydrolysis reactor is phase separated and the organic phase passes through one or more distillation columns to produce MMA product and light and/or heavy byproducts. In another embodiment, hydrolysis could be conducted within the distillation column itself.

Preferably, oxygen concentration at a reactor outlet is at least 1 mol %, more preferably at least 2 mol %, even more preferably at least 2.5 mol %, still more preferably at least 3 mol %, yet more preferably at least 3.5 mol %, even yet more preferably at least 4 mol %, and most preferably at least 4.5 mol %, based on the total volume of the gas stream exiting the reactor. Preferably, the oxygen concentration in a gas stream exiting the reactor is no more than 7.5 mol %, preferably no more than 7.25 mol %, preferably no more than 7 mol %, based on the total amount of the gas stream exiting the reactor.

One preferred embodiment of the fixed bed reactor for oxidative esterification is a trickle bed reactor, which contains a fixed bed of catalyst and passes both the gas and liquid feeds through the reactor in the downward direction. In trickle flow, the gas phase is the continuous fluid phase. Thus, the zone at the top of the reactor, above the fixed bed, will be filled with a vapor phase mixture of nitrogen, oxygen, and the volatile liquid components at their respective vapor pressures. Under typical operating temperatures and pressures (50-90 °C and 60-300 psig (400-2000 kPa)), this vapor mixture is inside the flammable envelope if the gas feed is air. Thus, only an ignition source would be required to initiate a deflagration, which could lead to loss of primary containment and harm to the physical infrastructure and personnel in the vicinity. In order to address process safety considerations, a means to operate a trickle bed reactor while avoiding a flammable headspace atmosphere is operation with a gas feed containing a sufficiently low oxygen mole fraction to ensure the oxygen concentration in the vapor headspace is below the limiting oxygen concentration (LOC).

Knowledge of the LOC is required for the fuel mixture, temperature, and pressure of concern. Since the LOC decreases with increasing temperature and pressure, and given that methanol gives a lower LOC than the other two significant fuels (methacrolein and methyl methacrylate), a conservative design chooses a feed oxygen to nitrogen ratio that ensures a composition with less than the LOC at the highest expected operating temperature and pressure. For example, for a reactor operated at up to 100 °C and 275 psig (2 MPa), the feed oxygen concentration in nitrogen should not exceed 7.4 mol %.

### EXAMPLES

### Examples 1-6—Semi Batch Slurry Reactor Operation

A 10 wt % methacrolein in methanol solution was prepared in advance. Approximately 1 gram of catalyst and 170 g of the methacrolein-methanol solution were placed in a 300 mL stirred reactor containing baffles and a gas induction propeller. The reactor was sealed and simultaneous mixing, pressurization, and heating began. The impeller was set to 1150 rpm and the reactor was pressurized to 100 psig by introducing a continuous flow of 100 sccm of 8% O2 in N2 gas which flowed through the reactor after pressurization was complete to a condenser. The reactor reached a reaction temperature of 80 °C in approximately 15 minutes at which time the reaction was defined to begin. Reactor aliquots were sampled every half hour for 2.5 to 5 hours using dip tubes equipped with in-line filters. Below Examples 1 through 6 were tested in this reactor system.

### Example #1

### Catalyst #1 Preparation:

A magnesium solution was first prepared by adding 51.4 g of magnesium nitrate hexahydrate and 5.5 g of 60% nitric acid in 100 mL of deionized water. Next approximately 100 g of Cariact Q50 silica was loaded in a 500 mL flask and 200 mL of magnesium solution was added while stirring. The slurry was stirred at 50 °C overnight, followed by filtration and the isolated material was dried in a vacuum oven at 80 °C for 1 h and then calcined at 500 °C overnight to produce a Mg modified silica. Approximately 25 g of Mg modified silica was added to 85 mL deionized water and the resulting slurry was heated to 90 °C while stirring. A second solution containing 0.453 g of hydrogen tetrachloroaurate (III) trihydrate, 1.255 g of cobalt nitrate hexahydrate and 85 mL of deionized water were prepared and then added to the slurry while stirring over a 30 min period and the resulting slurry was then stirred for another 1 hour. Solid materials were isolated from the slurry by centrifugation. The solids were then washed with a total of 300 mL of deionized water. In each washing the sample was mixed with 100 mL water for 5 min, followed by isolating the solid materials by centrifugation. The washed material was then dried at 105 °C for 10 h in air and then calcined at 450 C for 5 h with a ramping rate 5 °C/min.

### Example #2

### Catalyst #2 Preparation:

A slurry was prepared with 15 g of Siralox (Siralox 1.5/140), 200 mL deionized water, 0.352 g of hydrogen tetrachloroaurate (III) trihydrate, and 4.65 g of cerium nitrate hexahydrate. The slurry was then heated up to 45 °C and stirred for 1 hour prior to adjusting the pH to 8 by adding 1 M sodium carbonate dropwise. The slurry was stirred for another 2 hours at 45 °C before filtration. The resulting solids were washed by 500 mL deionized water during filtration, followed by drying under vacuum overnight at ambient temperature. The dried solids were then calcined in air in a box oven with an air flow of 40 L/min at 400 °C for 4 hours with a ramping rate of 5 °C/min.

### Example #3

### Catalyst #3 Preparation:

A slurry was prepared with 15 g of Siralox (Siralox 1.5/140), 200 mL deionized water, 0.456 g of hydrogen tetrachloroaurate (III) trihydrate and 1.16 g of ammonium perrhenate. The slurry was heated up to 60 °C and stirred for 1 hour prior to adjusting pH adjustment to 8 by adding 1 M sodium carbonate dropwise. The slurry was stirred for another 2 hours at 60 °C before filtration. The resulting solids were washed by 500 mL deionized water while filtering, followed by drying under vacuum overnight at ambient temperature. The dried solids were then calcined in air in a box oven with an air flow 40 L/min at 400 °C for 4 h with a ramping rate of 5 °C/min.

### Example #4

### Catalyst #4 Preparation:

A slurry was prepared with 15 g of Siralox (Siralox 1.5/140), 200 mL deionized water, 0.456 g of hydrogen tetrachloroaurate (III) trihydrate and 1.14 g of ammonium tungstate. The slurry was heated up to 60 °C and stirred for 1 hour prior to adjusting the pH to 8 by adding 1 M sodium carbonate dropwise. The slurry was stirred for another 2 hours at 60 °C before filtration. The resulting solids were washed by 500 mL deionized water while filtratering, followed by drying under vacuum overnight at ambient temperature. The dried solids were then calcined in air in a box oven with an air flow 40 L/min at 400 °C for 4 h with a ramping rate of 5 °C/min.

### Example #5

### Catalyst #5 Preparation:

A slurry was prepared with 15 g of Siralox (Siralox 1.5/140), 200 mL deionized water, 0.456 g of hydrogen tetrachloroaurate (III) trihydrate and 0.737 g of cadmium chloride. The slurry was heated up to 60 °C and stirred for 1 hour prior to adjusting the pH to 8 by adding 1 M sodium carbonate dropwise. The slurry was stirred for another 2 hours at 60 °C before filtration. The resulting solids were washed by 500 mL deionized water while filtratering, followed by drying under vacuum overnight at ambient temperature. The dried solids were then calcined in air in a box oven with an air flow 40 L/min at 400 °C for 4 h with a ramping rate of 5 °C/min.

### Example #6

### Catalyst #6 Preparation:

A slurry was prepared with 15 g of Siralox (Siralox 1.5/140), 200 mL deionized water, 0.456 g of hydrogen tetrachloroaurate (III) trihydrate and 0.87 g of zinc acetate dehydrate. The slurry was heated up to 60 °C and stirred for 1 hour prior to adjusting the pH to 8 by adding 1 M sodium carbonate dropwise. The slurry was stirred for another 2 hours at 60 °C before filtration. The resulting solids were washed by 500 mL deionized water while filtratering, followed by drying under vacuum overnight at ambient temperature. The dried solids were then calcined in air in a box oven with an air flow 40 L/min at 400 °C for 4 h with a ramping rate of 5 °C/min.

### Example 7—Semi Batch Recycle Fixed Bed Reactor Operation

A feed solution of 150 g was prepared comprising 10 wt % methacrolein, 200 ppm inhibitor and a balance of methanol, and placed in a 300 ml reactor vessel which served as a gas disengagement vessel. The vessel liquid was maintained at a temperature of approximately 20 °C. The liquid feed was pumped at 7 mL/min from the gas-disengagement vessel into the bottom of the vertically-oriented fixed bed reactor. Air and nitrogen gas was mixed to obtain 8 mol % oxygen and mixed with the liquid feed prior to entering the fixed bed reactor. The fixed bed reactor was a jacketed ¼″ stainless steel tube maintained at 60 °C using an external heater. The reactor itself was packed with 2 mm glass beads to fill approximately 18 inches (45.7 cm) of the tube, then catalyst. The remaining void at the top of the reactor was filled with 3 mm glass beads. Liquid and gas exiting the top of the reactor were sent to a condenser and non-condensable gases were vented, while the liquid was recycled back into the gas-disengagement vessel. Example 7 was tested in this reactor system.

### Example #7

### Catalyst #7 Preparation:

Catalyst was prepared by incipient wetness impregnation of 10 g of Fuji Silysia Chemical, Ltd. CARIACT Q-10 support which had been previously modified to add 6.6 wt % Ti in the form of titanium oxides by a vendor. A solution consisting of 0.39 g sodium gold thiosulfate, 0.4 g of mercaptosuccinic acid, and 0.12 g of citric acid monohydrate in 10 g of deionized water. The catalyst was then placed inside a box oven with constant air purging of 50 L/hr at ambient temperature for 1 hour and then ramped at 5 °C/min to 400 °C and calcined for 4 hours.

### Example 8—Single Pass Fixed Bed Reactor Operation

A feed consisting of 20 wt % methacrolein, 200 ppm polymerization inhibitor, and a balance of methanol was fed at a rate of 40 g/hr to a ⅜″ (9.5 mm) stainless steel tubular reactor containing a short front section of borosilicate glass beads followed by 5 g of catalyst. A gas containing 8% oxygen in nitrogen was also feed to the reactor at a rate sufficient to obtain 4.5% 02 in the vent. The reactor was operated at 60 °C and 160 psig (1200 kPa). The product of the reactor was sent to a liquid-vapor separator and the vapor was sent to a condenser with liquid going to the reactor exit and non-condensable gases going to the vent. Example 8 was tested in this reactor system.

### Example #8

### Catalyst #8 Preparation:

Catalyst was prepared by the incipient wetness technique using 20 g of Fuji Silysia Chemical, Ltd. CARiACT Q-10 support as the starting material and adding 10.5 g of titanium isopropoxide along with 3 g of glacial acetic acid in very small droplets in rotating equipment to ensure even distribution of the solution to the support material. The solution was at 40 C when added. The modified support material was then dried under vacuum at 60 C for 4 hrs and calcined in air at ambient pressure by ramping the temperature at 5 C/min from ambient to 125 C, holding for 1 hr and then ramping at 5 C/min up to 250 C and held for 1 hr, then ramped at 5 C/min to 350 C and held for 1 hr and finally ramped at 5 C/min to 450 C and held for 4 hrs. Gold was then added to the support by incipient wetness technique utilizing 0.83 g of sodium aurothiosulfate in 10 g of deionized water at 40 C. The resulting catalyst was dried and calcined in air using the same heating profile as above.

### Examples 9-11—Semi Batch Slurry Reactor Operation

A 10 wt % methacrolein in methanol solution was prepared in advance. Approximately 1 gram of catalyst and 170 g of the methacrolein-methanol solution were placed in a 300 mL stirred reactor containing baffles and a gas induction propeller. The reactor was sealed and simultaneous mixing, pressurization, and heating began. The impeller was set to 1150 rpm and the reactor was pressurized to 100 psig by introducing a continuous flow of 100 sccm of 8% O2 in N2 gas which flowed through the reactor after pressurization was complete to a condenser. The reactor reached a reaction temperature of 80 °C in approximately 15 minutes at which time the reaction was defined to begin. Reactor aliquots were sampled every half hour for 2.5 to 5 hours using dip tubes equipped with in-line filters. Below examples 9 through 11 were tested in this reactor system.

### Example #9

### Catalyst #9 Preparation:

As a first step, SiC support promoted with Zr was prepared. The support was prepared by impregnation-evaporation using a RotaVap. A portion of 10 g of SiC extrudates, sourced from SiCat, were loaded in a round bottom flask and 22 ml of a 0.5 M Zirconium(IV) oxynitrate hydrate in deionized water solution was added. The flask was placed on a RotaVap. Water was removed under vacuum at continuous flask rotation at 50 °C. The material was dried under vacuum for 30 min at 50 °C and then calcined in static air in a box oven using the following procedure: the sample was ramped from ambient conditions at 3 °C/min and dried at 120 °C for 2 hr followed by ramping at 2 °C/min and calcination at 600 °C for 4 hr

As a second step, Au was added to the support material. A 1.5 wt % Au/Zr/SiC catalyst was prepared by incipient wetness impregnation followed by drying and calcination in air using a box oven. A 0.1988 M solution was prepared by placing 3.877 g of sodium aurothiomalate(I) in a volumetric flask and filling with deionized water to a volume of 50 ml. The flask was gently stirred until a transparent yellow solution was formed. Next an impregnation solution was prepared by adding 1.7 ml of deionized water to 38.3 ml of the 0.1988 M sodium aurothiomalate(I) solution and 5 g of the Zr—SiC support were impregnated with 2 ml of this impregnation solution dropwise until the incipient wetness point for a 1.5 wt % Au catalyst was reached. The impregnated material was dried and then calcined in static air in a box oven using the following procedure: the sample was ramped from ambient conditions at 5 °C/min and dried at 120 °C for 1 hr followed by ramping at 5 °C/min and calcination at 300 °C for 4 hr.

### Example #10

### Catalyst #10 Preparation:

As a first step, SiC support promoted with Ti was prepared. The support was prepared by impregnation-evaporation using a RotaVap. A portion of 10 g of SiC extrudates, sourced from SiCat, were loaded in a round bottom flask and 5.3 ml of 50 wt % titanium(IV) bis(ammonium lactato)dihydroxide solution was added. The flask was placed on a RotaVap. Water was removed under vacuum at continuous flask rotation at 50 °C. The material was dried under vacuum for 30 min at 50 °C and then calcined in static air in a box oven using the following procedure: the sample was ramped from ambient conditions at 3 °C/min and dried at 120 °C for 2 hr followed by ramping at 2 °C/min and calcination at 600 °C for 4 hr.

As a second step, Au was added to the support material. A 1.5 wt % Au/Ti/SiC catalyst was prepared by incipient wetness impregnation followed by drying and calcination in air using a box oven. A 0.1988 M solution was prepared by placing 3.877 g of sodium aurothiomalate(I) in a volumetric flask and filling with deionized water to a volume of 50 ml. The flask was gently stirred until a transparent yellow solution was formed. Next an impregnation solution was prepared by adding 1.7 ml of deionized water to 38.3 ml of the 0.1988 M sodium aurothiomalate(I) solution and 5 g of the Ti—SiC support were impregnated with 2 ml of this impregnation solution dropwise until the incipient wetness point for a 1.5 wt % Au catalyst was reached. The impregnated material was dried and then calcined in static air in a box oven using the following procedure: the sample was ramped from ambient conditions at 5 °C/min and dried at 120 °C for 1 hr followed by ramping at 5 °C/min and calcination at 300 °C for 4 hr.

### Example #11

### Catalyst #11 Preparation:

As a first step, SiC support promoted with La was prepared. The support was prepared by impregnation-evaporation using a RotaVap. A portion of 10 g of SiC extrudates, sourced from SiCat, were loaded in a round bottom flask and 17.7 ml of 0.623 M Lanthanum (III) nitrate hexahydrate solution was added. The flask was placed on a RotaVap. Water was removed under vacuum at continuous flask rotation at 50 °C. The material was dried under vacuum for 30 min at 50 °C and then calcined in static air in a box oven using the following procedure: the sample was ramped from ambient conditions at 3 °C/min and dried at 120 °C for 2 hr followed by ramping at 2 °C/min and calcination at 600 °C for 4 hr.

As a second step, Au was added to the support material. A 1.7 wt % Au/La/SiC catalyst was prepared by incipient wetness impregnation followed by drying and calcination in air using a box oven. A 0.1988 M solution was prepared by placing 3.877 g of sodium aurothiomalate(I) in a volumetric flask and filling with deionized water to a volume of 50 ml. The flask was gently stirred until a transparent yellow solution was formed. Next an impregnation solution was prepared by adding 1.7 ml of deionized water to 38.3 ml of the 0.1988 M sodium aurothiomalate(I) solution and 5 g of the La—SiC support were impregnated with 2 ml of this impregnation solution dropwise until the incipient wetness point for a 1.7 wt % Au catalyst was reached. The impregnated material was dried and then calcined in static air in a box oven using the following procedure: the sample was ramped from ambient conditions at 5 °C/min and dried at 120 °C for 1 hr followed by ramping at 5 °C/min and calcination at 300 °C for 4 hr.

The results for Examples 1-11 are shown below in Table 1.

TABLE 1

| | | | | | | Activity as | |
|---|---|---|---|---|---|---|---|
| | | | | | | Space-Time- | Normalized |
| Catalyst | | | | Gold | Promotor | Yield (mol | MMA |
| Example | Catalyst | Prep | | Load⁺ | Load⁺ | MMA/kg | Selectivity⁺⁺ |
| # | Description | Method* | Eggshell** | (wt %) | (wt %) | cat-hr) | (%) |
| 1 | AuCo/Mg/SiO₂ | 1 | NA | 0.8 | Co (0.55), | 18.2 | 91.7 |
| | | | | | Mg (1.9) | | |
| 2 | AuCe/SiO₂ | 1 | NA | 0.95 | Ce (8.3) | 15 | 97.8 |
| 3 | AuRe/SiO₂ | 1 | NA | 1.43 | Re (0.0031) | 27.1 | 96.4 |
| 4 | AuW/SiO₂ | 1 | NA | 1.28 | W (1.06) | 35.6 | 91.2 |
| 5 | AuCd/SiO₂ | 1 | NA | 0.58 | Cd (1.06) | 11.4 | 88.7 |
| 6 | AuZn/SiO₂ | 1 | NA | 0.53 | Zn (1.74) | 20.3 | 78.7 |
| 7 | Au/Ti/SiO₂ | 2 | Yes | 1.1 | Ti (6.6) | 4.2 | 99 |
| 8 | Au/Ti/SiO₂ | 3 | Yes | 1.5 | Ti (6) | 4.9 | 98.9 |
| 9 | Au/Zr/SiC | 1, 2 | Yes | 1.5 | Zr (6.9) | 27.3 | 99 |
| 10 | Au/Ti/SiC | 1, 2 | Yes | 1.5 | Ti (4.0) | 30.6 | 99 |
| 11 | Au/La/SiC | 1, 2 | NA | 1.7 | La (11.5) | 29.3 | 99 |

*The preparation methods include: (1) submersion impregnation, (2) incipient wetness impregnation, and (3) spray or small droplet impregnation
**Eggshell catalyst is defined as having at least 90 wt % gold content in the outer 40 volume % of the catalyst pellet. NA is not available.
⁺Estimated by energy-dispersive spectroscopy (EDS) with a scanning electron microscope (SEM) or from the preparation method in combination with comparison to other catalysts when available.
⁺⁺The normalized MMA selectivity is the percent MMA among products originating as methacrolein reactant.

1. A process for making a catalyst for oxidative esterification of methacrolein to methyl methacrylate comprising:
providing a support, wherein the support comprises a material selected from the group consisting of an oxide of silicon, a carbide of silicon, a metal oxide, and a metal carbide;
providing particles of at least one oxide of a metal on a surface of the support, wherein the metal is selected from the group consisting of aluminum, titanium, lanthanides, zirconium, nickel, cobalt, zinc, tellurium, antimony, bismuth, rhenium, tungsten, alkali metals, and alkaline earth metals,
contacting the support with a gold salt,
heating the support at a temperature ranging from 50 °C to 600 °C for a time ranging from at least 0.1 h to 48 h to convert the gold salt to gold nanoparticles having an average diameter less than 12 nm and a standard deviation of +/−4 nm, wherein at least 75% by number of the gold nanoparticles are positioned within 20 nm of a particle of the oxide of the metal.

2. The process of claim 1, wherein the support has an average diameter ranging from 50 nm to 500 μm, and the step of providing the support comprises producing the support by a method selected from the group of reacting to form the support followed by drying, spray drying, precipitation followed by filtration and/or centrifugation, and crushing.

3. The process of claim 1, wherein the support has an average diameter ranging from greater than 500 μm to 10 mm, and the step of providing the support comprises producing the support by a method of extrusion or pelletization.

4. The process of claim 1, wherein the step of heating the support comprises heating the support at a temperature ranging from 150 °C to 500 °C for a time ranging from at least 0.1 h to 48 h in the presence of an oxygen-containing gas.

5. The process of claim 1, wherein the step of heating the support comprises heating the support in the presence of a reducing gas comprising at least 0.1% by volume of a reducing agent.

6. The process of claim 1, wherein the step of heating the support comprises heating the support in an inert atmosphere.

7. The process of claim 1, wherein the step of heating the support comprises heating the support in the presence of a solvent and a reductant, wherein the ratio of reductant to solvent is at least 0.01.

8. The process of claim 1, wherein contacting the support with a gold salt comprises a method selected from the group consisting of:
impregnating the support with a solution comprising the gold salt;
dip coating the support with a solution comprising the gold salt;
spray coating the support with a solution comprising the gold salt; and
sequentially impregnating the support by impregnating the support with a first solution to fill at least 80% of the volume of any pores in the support with a pore filling agent, followed by impregnating the support with a second solution comprising the gold salt.

9. The process of claim 1, wherein at least 75% by weight of the total weight of the gold particles are within an outer 50% of the volume of the catalyst.

10. The process of claim 1, wherein the oxide of the metal comprises an oxide of nickel or an oxide of titanium.

11. The catalyst of claim 1, wherein the support comprises an oxide of silicon and an oxide of titanium.

12. A method for preparing methyl methacrylate from methacrolein and methanol; said method comprising contacting in a reactor a mixture comprising methacrolein, methanol and oxygen in the presence of a catalyst according to claim 1.
